(19) United States
(12) Patent Application Publication
(10) Pub. No.: US 2025/0090430 A1
(43) Pub. Date: Mar. 20, 2025

(54) DEODORANT/ANTIPERSPIRANT STICK COMPOSITIONS

(71) Applicant: Conopco, Inc., d/b/a UNILEVER, Englewood Cliffs, NJ (US)

(72) Inventors: Huajin JIN, Shanghai (CN); Xiaoke LI, Shanghai (CN); Renjiang LIU, Shanghai (CN); Weichong WANG, Shanghai (CN)

(73) Assignee: Conopco, Inc., d/b/a UNILEVER, Englewood Cliffs, NJ (US)

(21) Appl. No.: 18/294,190

(22) PCT Filed: Aug. 26, 2022
(86) PCT No.: PCT/EP2022/073744

§ 371 Date: Feb. 1, 2024

(30) Foreign Application Priority Data
Sep. 3, 2021 (WO) PCT/CN2021/116349
Oct. 20, 2021 (EP) 21203600.8

(51) Int. Cl. A61K 8/06; A61Q 15/00

(52) U.S. Cl. CPC A61K 8/062; A61Q 15/00; A61K 2800/5422; A61K 2800/805

(57) ABSTRACT

The present invention relates to solidified, oil-in-water dispersion or emulsion deodorant or antiperspirant stick compositions. Disclosed is a deodorant or an antiperspirant stick composition in the form of an oil-in-water dispersion or emulsion, comprising: i) at least one lipophilic material having a melting point higher than 50° C.; ii) at least one nonionic emulsifier having a HLB value from 1 to 11 within an oil-in-water emulsifier mixture with an average HLB value in the range of from 6.8 to 11; iii) an aqueous cosmetically acceptable carrier; wherein said composition comprises at least 8% of said lipophilic material by weight of the composition; wherein said composition comprises higher than 50 wt % of water by weight of the composition; wherein said lipophilic material has a straight alkyl chain comprising at least 20 carbon atoms.

### FIELD OF THE INVENTION

The present invention relates to solidified, oil-in-water dispersion or emulsion deodorant or antiperspirant stick compositions and their methods of manufacture, especially to such compositions containing a high amount of water.

### BACKGROUND OF THE INVENTION

Deodorant or antiperspirant stick compositions are typically characterised by being integral and self-supporting, commonly in the form of a rod or bar, usually dispensed from a tubular container having an open end and provided with a platform that is moveable towards the open end. Some deodorant or antiperspirant stick compositions are anhydrous, whilst others are aqueous compositions in the form of an emulsions. In such emulsion compositions, the deodorant or antiperspirant active is commonly dissolved in the aqueous phase which is typically dispersed in an oil continuous phase to form a water-in-oil dispersion or emulsion. The water-soluble active ingredients are present in the inner, dispersed phase and, following application, must first migrate through the outer, lipophilic layer in order to reach their site of action on the skin. The known water-in-oil emulsion sticks thus have disadvantages which are similar to those of anhydrous suspension sticks with regard to the availability of active ingredient. There is a need to develop deodorant or antiperspirant stick compositions based on oil-in-water dispersion or emulsion.

The present invention may relate to compositions that contain antiperspirant actives. These actives are added to compositions to reduce perspiration upon topical application of the compositions to the body, particularly to the underarm regions of the human body viz. the axilla, and sometimes even on the upper part of the body near the chest. Usually, conventional antiperspirant actives are salts of certain metals having an astringent effect, such as the salts of aluminium and/or zirconium. Since antiperspirants are used regularly, and have been used for decades, there is an ever-increasing need to develop alternative antiperspirant actives which are equally efficacious. US 2009/0010864 A1 discloses deodorant or antiperspirant sticks based on an ethanol-containing oil-in-water dispersion/emulsion. It comprises 5% up to less than 50% by weight of water.

### SUMMARY OF THE INVENTION

The present invention relates to deodorant or antiperspirant stick formulations which is in form of oil-in-water dispersion or emulsion having high quantity of water.

The present invention addresses a problem found with the performance of deodorant or antiperspirant stick compositions that are in the form of oil-in-water dispersion or emulsion.

The deodorant or antiperspirant stick composition that are in the form of oil-in-water dispersion or emulsion have low amount of water in order to have stable stick composition with acceptable appearance. The present invention provides a deodorant or an antiperspirant stick formulations which is in form of oil-in-water dispersion or emulsion having relatively high levels of water. The benefit of the present invention is that relatively high levels of water can be incorporated in the stick composition to deliver coolness or freshness after application.

A further benefit of the present invention is that the present invention provides an oil-in-water dispersion or emulsion comprising specific emulsifier and lipophilic material which can lead to a reduction in perspiration. Once the oil-in-water emulsion is applied, gradual evaporation of the water of emulsion induces the breakdown of the emulsion and the aggregation or coalescence of the emulsifier and lipophilic material which are in solid form thereby form an aggregation/precipitation and which thereby provides antiperspirant benefits.

It is a further object of the present invention to provide an oil-in-water deodorant or antiperspirant stick formulation that exhibits desirable sensory properties either on application or thereafter.

It is a further object of the present invention to provide an oil-in-water deodorant or antiperspirant stick formulation that delivers good antiperspirancy efficacy.

It is a yet another object of certain of the embodiments of the invention to provide an oil-in-water deodorant or antiperspirant stick formulation which results in reduced visible marks on clothing following application. Such visible marks are typically seen as yellow staining, on fabrics worn in proximity to the site of application of the composition.

In accordance with a first aspect is disclosed deodorant or antiperspirant stick composition in the form of an oil-in-water dispersion or emulsion, comprising:
i) at least one lipophilic material having a melting point higher than 50° C.;
ii) at least one nonionic emulsifier having a HLB value from 1 to 11 within an oil-in-water emulsifier mixture with an average HLB value in the range of from 6.8 to 11;
iii) an aqueous cosmetically acceptable carrier; wherein said composition comprises at least 8% of said lipophilic material by weight of the composition; wherein said composition comprises higher than 50 wt % of water by weight of the composition;
wherein said lipophilic material has a straight alkyl chain comprising at least 20 carbon atoms.

In accordance with a second aspect is disclosed a process for the preparation of a deodorant or an antiperspirant stick composition in accordance with the first aspect of the invention.

In accordance with a third aspect is disclosed a method of reducing malodour or perspiration comprising a step of topical application of the composition of the first aspect on to the desired skin surface.

In accordance with a third aspect is disclosed use of the composition of the first aspect for reduction of bodily malodour or perspiration.

All other aspects of the present invention will more readily become apparent upon considering the detailed description and examples which follow.

Except in the examples, or where otherwise explicitly indicated, all numbers in this description indicating amounts of material or conditions of reaction, physical properties of materials and/or use may optionally be understood as modified by the word “about”.

All amounts are by weight of the antiperspirant composition, unless otherwise specified.

It should be noted that in specifying any ranges of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

### DETAILED DESCRIPTION OF THE INVENTION

By “a deodorant or an antiperspirant composition” as used herein, is meant to include a composition for topical application to the skin of mammals, especially humans. Such a composition is preferably of the leave-on type. By a leave-on composition is meant a composition that is applied to the desired skin surface and left on for one minute to 24 hours after which it may be wiped or rinsed off with water, usually during the regular course of personal washing. The composition may also be formulated into a product which is applied to a human body for improving the appearance, cleansing, odor control or general aesthetics. The composition of the present invention can be in the form of a liquid, lotion, cream, gel or stick form and may be delivered through a roll-on device or using an aerosol can which contains a propellant. “Skin” as used herein is meant to include skin on any part of the body (e.g., neck, chest, back, arms, underarms, hands, legs, buttocks and scalp) especially the underarms.

Stick compositions are usually available in the form of a firm solid or a soft solid. Firm solids, as the name indicates, are harder and can be directly applied by way of an applicator, for example, to the underarms. Soft solids also need an applicator which is similar to the firm solids, the difference being that the soft solids are softer and the applicator needs to be designed in order to permit extrusion of the solids through a cap member comprising plurality of orifices and the extruded composition can then be applied to the underarms.

It is preferred that the stick composition of the present invention is antiperspirant stick composition.

### HLB Value

“HLB value” refers to a value defining the affinity of a surfactant for water or oil. HLB numbers can be calculated for nonionic surfactants, and these surfactants have numbers ranging from 0-20.

If it's a mixture of surfactants, the HLB value can be calculated as below:

$$HLBmix = \frac{( {C}_{1} \times {HLB}_{1} ) + ( {C}_{2} \times {HLB}_{2} ) + ( {C}_{3} \times {HLB}_{3} ) …}{{C}_{Total}}$$

$$C 1 , C 2 , C 3 … = weight proportion of each surfactant , wt \%$$

$${C}_{Total} = C 1 + C 2 + C 3 + …$$

$$HLB 1 , HLB 2 , HLB 3 … = HLB value of each surfactant$$

### Melting Point

“Melting point” refers to the temperature at which the solid and liquid forms of a pure substance can exist in equilibrium. “Melting point” for the purpose of the present invention is specified at a standard pressure such as 1 atmosphere.

### Oil-In-Water Dispersion or Emulsion

Oil-in-water emulsion comprises an oil phase and a water phase wherein oil phase is the dispersed phase and water is the dispersion medium.

Oil-in-water dispersion comprises an oil phase and a water phase wherein oil is dispersed as droplets of oil phase in the water phase.

Deodorant or antiperspirant compositions of the present invention comprise an oil-in-water dispersion or emulsion.

### Emulsifier

The stick composition in accordance with this invention comprises a nonionic emulsifier. And such a nonionic emulsifier has an HLB value of from 1 to 11 within an oil-in-water emulsifier mixture with an average HLB value in the range of from 6.8 to 11, preferably from 7 to 11, more preferably from 7.5 to 10.5.

It is preferred that such nonionic emulsifier is selected from sorbitan ester, fatty alcohol ethoxylate or fatty acid ethoxylates, sucrose ester, glyceryl ester or polyglyceryl ester, polyol ester, and cetearyl glucoside.

When the nonionic emulsifier is a sorbitan ester, it is preferably selected from Tween 61 (polyoxyethylene sorbitan monostearate), Tween 65 (polyoxyethylene sorbitan tristearate), Tween 81 (polyoxyethylene sorbitan monooleate), Tween 85 (polyoxyethylene sorbitan trioleate), PEG-40 sorbitan peroleate, Span 20 (sorbitan monolaurate), Span 40 (sorbitan monopalmitate), Span 60 (sorbitan monostearate), Span 65 (sorbitan tristearate), Span 80 (sorbitan monooleate), Span 83 (sorbitan sesquioleate), Span 120 (sorbitan isostearate).

When the nonionic emulsifier is a fatty alcohol ethoxylate or fatty acid ethoxylate, it is preferably selected from steareth-2, ceteth-2, isoceteth-20, laureth-4, oleth-2, ceteareth-20.

When the nonionic emulsifier is a glyceryl ester or polyglyceryl ester, it is preferably selected from PEG-25 hydrogenated castor oil, glyceryl Laurate, glyceryl stearate, polyglyceryl-2-distearate, polyglyceryl-2 stearate, polyglyceryl-3 distearate.

When the nonionic emulsifier is a polyol ester, it is preferably selected from glycol stearate, PEG-4 dilaurate, PEG-30 dipolyhydroxystearate, PEG-20 methyl glucose sesquistearate.

The deodorant or antiperspirant composition in accordance with this invention comprises at least 2 wt %, preferably from 2 to 20%, more preferably from 2.5 to 10 wt %, and furthermore preferably from 3 to 8 wt %, and furthermore preferably from 3.5 to 5 wt % the emulsifier by weight of the composition.

In order to assist the formation and stability of an emulsion or dispersion, it is preferred that deodorant or antiperspirant composition in accordance with this invention additionally comprises a nonionic emulsifier having a HLB value from 7 to 20, which is the second nonionic emulsifier in the composition.

It is preferred that the additional nonionic emulsifier is selected from sorbitan ester, sucrose ester, fatty alcohol ethoxylate or fatty acid ethoxylates, glyceryl ester or polyglyceryl ester, polyol ester, cetearyl glucoside, and sucrose stearate.

When the additional nonionic emulsifier is a sorbitan ester, it is preferably selected from Tween 40 (Polyoxyethylene sorbitan monopalmitate), Tween 20 (Polyoxyethylene sorbitan monolaurate), Tween 21(Polyoxyethylene sorbitan monolaurate), Tween 60 (Polyoxyethylene sorbitan monostearate), Tween 61 (Polyoxyethylene sorbitan monostearate), Tween 65 (Polyoxyethylene sorbitan tristearate), Tween 80 (Polyoxyethylene sorbitan monooleate), Tween 81(Polyoxyethylene sorbitan monooleate), Tween 85 (Polyoxyethylene sorbitan trioleate), PEG-40 sorbitan peroleate, PEG-80 sorbitan laurate, Span 20 (sorbitan monolaurate) 20, Span 40 (sorbitan monopalmitate), Span 60 (sorbitan monostearate), Span 80 (sorbitan monooleate), Span 85 (sorbitan trioleate).

When the additional nonionic emulsifier is a fatty alcohol ethoxylate or fatty acid ethoxylate, it is preferably selected from ceteareth-20, ceteth-10, ceteth-20, isoceteth-20, isosteareth-20, oleth-10, laureth-4, oleth-20, steareth-20, steareth-21, laureth-23.

When the additional nonionic emulsifier is a glyceryl ester or polyglyceryl ester, it is preferably selected from PEG-25 hydrogenated castor oil, PEG-60 almond glycerides, polyglyceryl-10 monopalmitate, polyglyceryl-10 stearate. The polyol ester can be selected from PEG-100 stearate, PEG-8 laurate, PEG-40 stearate, PEG-8 laurate.

It is preferred that the composition in accordance with this invention comprises from 0.5 to 20 wt %, and more preferably from 1 to 10 wt %, and furthermore preferably from 2 to 5 wt % such emulsifier by weight of the composition.

### Lipophilic Material

“Lipophilic material” for the purpose of the present invention means a material which tends to combine with or dissolve in lipids, oils or fats.

Without wishing to be bound by theory it is believed that the lipophilic material has to be in solid form upon application of the composition to the skin, e.g., the underarms. Therefore, it is required that the lipophilic material has relatively high melting point.

The lipophilic material in accordance with this invention has a melting point greater than 50° C., preferably greater than 55° C., more preferably greater than 60° C.

The lipophilic material in accordance with the present invention has a straight alkyl chain comprising at least 20 carbon atoms, preferably comprising 20-40 carbon atoms, more preferably comprising 20-30 carbon atoms.

It is preferred that the lipophilic material is a fatty alcohol, a fatty acid, a fatty ester, a wax or a mixture thereof.

When the lipophilic material is a fatty alcohol, it is preferred that the fatty alcohol has a straight alkyl chain comprising 20 to 40 carbon atoms, more preferably comprising 20-30 carbon atoms. It can be a fatty alcohol selected from arachidyl alcohol (C20), behenic alcohol (C22), policsanol (C28) and triacontanol (C30).

When the lipophilic material is a fatty acid, it is preferred that the fatty acid has a straight alkyl chain comprising 20-40 carbon atoms, more preferably comprising 20-30 carbon atoms. It can be a fatty acid selected from behenic acid (C22), eicosanoic acid(C20) and tetracosanoic acid (C24).

When the lipophilic material is a wax, it is preferred that the wax has a straight alkyl chain comprising 20-40 carbon atoms, more preferably comprising 20-30 carbon atoms.

When the lipophilic material is a fatty ester, it is preferred that fatty ester has a straight alkyl chain comprising 20-40 carbon atoms, more preferably comprising 20-30 carbon atoms. It can be selected from tribehenin, C20-36 fatty acid ester of triglyceride, C20-36 fatty acid ester of glycol, one ester of a saturated monohydric C20-C40 alcohol, one ester of a saturated C20-C36-monocarboxylic acid, C20-C40 dialkyl esters of dimer acids. It is most preferred that the fatty ester is tribehenin.

The composition in accordance with this invention comprises at least 8 wt % of such lipophilic material by weight of the composition, preferably at least 10%, more preferably at least 12% of such lipophilic material by weight of the composition. It is preferred that the composition in accordance with this invention comprises from 8 to 50 wt %, and more preferably from 10 to 40 wt %, and furthermore preferably from 12 to 35 wt % the lipophilic material, and most preferably from 12 to 20 wt %, based on total weight of the composition.

Without wishing to be bound by theory the inventors believe that once the oil-in-water dispersion or emulsion is applied, gradual evaporation of the water induces the breakdown of the dispersion or emulsion and the aggregation or coalescence of the emulsifier and lipophilic material which are in solid form thereby form an aggregation/precipitation and which thereby provides antiperspirant benefits.

Compositions in accordance with this invention may advantageously comprise an additional antiperspirant active. It may comprise conventional antiperspirant active which comprises aluminium or zirconium or zinc. Antiperspirant active which comprises aluminium or zirconium is preferably selected from aluminium/zirconium halides and halohydrate salts, aluminum-zirconium tetrachlorohydrex glycine complex, aluminum-zirconium octachlorohydrex glycine complex, aluminum-zirconium pentachlorohydrate, aluminum sesquichlorohydrate or mixtures thereof, more preferably aluminum sesquichlorohydrate or aluminium chlorohydrates. Antiperspirant active which comprises aluminium or zirconium can also be complexes based on the above-mentioned astringent aluminium and/or zirconium salts and the complex often employs a compound with a carboxylate group, and advantageously this is an amino acid. Zinc based antiperspirant active is preferably selected from zinc oxide, zinc hydroxide, zinc hydroxide ions with counter ions, and zinc ions with counter ions. Counter ions may include halides and amino acid salt.

However, the composition of the present invention is preferably free from antiperspirant active which comprises aluminium or zirconium or zinc.

Non-aluminum and non-zirconium, non-zinc antiperspirant actives may be present to augment or supplement the antiperspirant activity of the non-thermoplastic polymeric material as disclosed. Non- aluminum or zirconium, or non-zinc antiperspirant active is preferably selected from glycerol monolaurate plus isostearyl alcohol, chitosan or a salt thereof with a weight average molecular weight of from 250 to 650 kDa, titanium compound chelated by alkanolamine with an acid and a polyhydric alcohol, or cholic acid derivative selected from a hydroxycholic acid or a salt thereof with a multivalent metal salt, such as water-soluble calcium salts and magnesium salts.

More preferably, the composition of the present invention is free from metal based antiperspirant active. The metal includes aluminum, zirconium, zinc, titanium, copper, gallium, stannum, Indium, hafnium, vanadium, cobalt.

### Water

In accordance with the present invention, the deodorant or antiperspirant composition comprise higher than 50% of water, preferably from 60% to 90%, more preferably from 60% to 85% of water by weight of the composition.

### pH of Compositions

It is preferred that pH of the composition of the present invention is preferably from 2 to 9, more preferably 3 to 7.

### Other Ingredients

Other components commonly included in conventional deodorant or antiperspirant compositions may also be incorporated in the compositions of the present invention. Such components include skin care agents such as emollients, humectants and skin barrier promoters; skin appearance modifiers such as skin lightening agents and skin smoothing agents; anti-microbial agents, in particular organic anti-microbial agents, and preservatives.

The composition of the invention comprises an aqueous cosmetically acceptable carrier. The term aqueous means that the composition of the invention comprises water as the main carrier or that water forms a major part of the carrier. In such cases, other solvents and ingredients other than water may also be present.

The hardness of the stick can be measured by a texture analyzer (Stable Micro Systems). A needle penetrated the solid emulsion for 10 mm with a speed of 2 mm/s, and the force curve is recorded as the hardness curve. The maximum force (kg) of the curve is determined as the hardness.

The stick composition of the invention has desired hardness of at least 0.015 kg, preferably at least 0.02 kg, more preferably at least 0.04 kg. Such sticks can demonstrate a combination of aesthetically desirable properties, including sensory benefits.

The composition of the present invention can comprise a wide range of other optional components. The CTFA Personal Care Ingredient Handbook, Second Edition, 1992, which is incorporated by reference herein in its entirety, describes a wide variety of non-limiting personal care and pharmaceutical ingredients commonly used in the skin care industry, which are suitable for use in the compositions of the present invention. Examples include: antioxidants, binders, biological additives, buffering agents, colorants, thickeners, polymers, astringents, fragrance, conditioners, exfoliating agents, pH adjusters, other than the ones already discussed earlier, preservatives, natural extracts, essential oils, skin sensates, skin soothing agents, and skin healing agents.

A preservative is a preferred additional component in compositions of the invention. A preservative serves to reduce or eliminate microbial contamination of compositions of the invention. Preservatives are typically employed at a total level of from 0.05 to 3%, preferably at from 0.1 to 2% and most preferably at from 0.4 to 1%.

Suitable preservatives for use with the present invention include 2-phenoxyethanol, polylysine, iodopropynyl butylcarbamate, C₁-C₃ alkyl parabens, sodium benzoate, caprylyl glycol and EDTA. Particularly preferred preservatives are 2-phenoxyethanol, iodopropynyl butylcarbamate, sodium benzoate, caprylyl glycol and EDTA and especially preferred are 2-phenoxyethanol and iodopropynyl butylcarbamate.

A preferred additional component of compositions of the invention is a fragrance. Suitable materials include conventional perfumes, such as perfume oils and also include so-called deo-perfumes, as described in EP 545,556 and other publications. Levels of incorporation are preferably up to 4% by weight, particularly from 0.1% to 2% by weight, and especially from 0.7% to 1.7% by weight.

An antimicrobial deodorant active is a preferred an additional component in compositions of the invention. Such components serve to reduce or eliminate body odour by reducing or otherwise impeding the function of microbes on the skin of the body responsible for malodour generation.

The antimicrobial deodorant active may also be a preservative for the composition.

When employed, the anti-microbial deodorant agent is typically incorporated into the composition at from 0.01% to 3% and particularly at from 0.03% to 0.5%.

Preferred anti-microbial deodorant agents have a minimum inhibitory concentration (MIC) of 1 mg·ml⁻¹ or less, particularly 200 μg·ml⁻¹ or less, and especially 100 μg·ml⁻¹ or less. The MIC of an anti-microbial agent is the minimum concentration of the agent required to significantly inhibit microbial growth. Inhibition is considered “significant” if an 80% or greater reduction in the growth of an inoculum of Staphylococcus epidermidis is observed, relative to a control medium without an anti-microbial agent, over a period of 16 to 24 hours at 37° C. Details of suitable methods for determining MICs can be found in “Antimicrobial Agents and Susceptibility Testing”, C. Thornsberry, (in “Manual of Clinical Microbiology”, 5^{th} Edition, Ed. A. Balows et al, American Society for Microbiology, Washington D.C., 1991). A particularly suitable method is the Macrobroth Dilution Method as described in Chapter 110 of above publication (pp. 1101-1111) by D. F. Sahm and J. A. Washington II. MICs of anti-microbials suitable for inclusion in the compositions of the invention are triclosan: 0.01-10 μg·ml⁻¹ (J. Regos et al., Dermatologica (1979), 158: 72-79) and farnesol: ca. 25 μg·ml⁻¹ (K. Sawano, T. Sato, and R. Hattori, Proceedings of the 17^{th} IFSCC International Conference, Yokahama (1992) p. 210-232). By contrast ethanol and similar alkanols have MICs of greater than 1 mg·ml⁻¹.

Suitable organic anti-microbials are bactericides, for example quaternary ammonium compounds, like cetyltrimethylammonium salts; chlorhexidine and salts thereof; and diglycerol monocaprate, diglycerol monolaurate, glycerol monolaurate, and similar materials, as described in “Deodorant Ingredients”, S. A. Makin and M. R. Lowry, in “Antiperspirants and Deodorants”, Ed. K. Laden (1999, Marcel Dekker, New York). More preferred anti-microbials for use in the compositions of the invention are polyhexamethylene biguanide salts (also known as polyaminopropyl biguanide salts), an example being Cosmocil CQ™ available from Zeneca PLC, preferably used at up to 1% and more preferably at 0.03% to 0.3% by weight; 2′,4,4′-trichloro,2-hydroxy-diphenyl ether (triclosan), preferably used at up to 1% by weight of the composition and more preferably at 0.05-0.3%; and 3,7,11-trimethyldodeca-2,6,10-trienol (farnesol), preferably used at up to 1% by weight of the composition and more preferably at up to 0.5%.

Other suitable organic antimicrobial agents are transition metal chelators, as described in WO01/52805, for example. Transitional metal chelators having a binding coefficient for iron(Ill) of greater than 10²⁶, for example diethylenetriaminepentaacetic acid and salts thereof are preferred.

### Process

The invention stick compositions can be made in a process comprising: in step 1,

making an aqueous phase by blending together the water and water-soluble ingredients, at least a fraction of which may be in the form of a pre-formed aqueous solution; in step 2, forming an oil phase comprising the lipophilic material and nonionic emulsifier, which normally entails heating to a temperature at which solidifying agent particles are no longer visible, commonly in the region of the melting point of the lipophilic material; and in a step 3, mixing the aqueous phase and the oil phase together at elevated temperature such that the ingredients remains in a liquid state, and preferably shear mixing the two phases, in the presence of an additional emulsifier; in step 4, cooling the mixture to 50° C. or below while homogenizing; in step 5, filling the resultant mixture into a dispenser whilst it is still mobile and there after cooling or allowing the composition to cool, initially until the mixture solidifies and then attains ambient temperature.

Steps 1 and 2 in the process described above can be carried out simultaneously or sequentially. In step 2, the lipophilic material is often heated to a temperature in the region of 70 to 80° C., preferably 70 to 75° C. Before the aqueous phase, which is prepared in step 1, is mixed with the oil phase in step 3, it preferably heated to temperature in the region of 60 to 80° C., more preferably 65 to 75° C. and most preferably 68 to 73° C. Using these elevated temperatures for the aqueous phase can aid the emulsification process. During step 3, the temperature is maintained at preferably 65 to 80° C., more preferably 65 to 75° C. and most preferably 66 to 74° C.

It is often convenient to incorporate ingredients which are not water-soluble, such as water-insoluble optional ingredients and/or the emulsifier in step 2. If desired, the solidifying agent, or a fraction of them, can be heated externally, for example melted, prior to introduction into the oil phase or emulsion.

If any temperature sensitive ingredients, such as certain fragrance components, are to be added to the compositions, it is often best to add these after step 3 and when the temperature has been reduced, e.g. to less than 70° C., such as from 40 to 50° C.

In a particular embodiment of the process described in the above paragraphs, a deodorant or an antiperspirant composition is manufactured by the steps of (i) making an aqueous phase by blending together water and water-soluble ingredients and heating said phase to a temperature of 60° C. or greater; (ii) forming an oil phase comprising a lipophilic material and nonionic emulsifier and heating said phase to a temperature of 70° C. or greater; (iii) with the aqueous phase and oil phase at the temperatures as indicated in steps (i) and (ii) respectively, mixing these phases together; (iv) cooling the mixture to 50° C. or below while homogenizing; (v) filling the resultant mixture into a dispenser whilst it is still mobile; and (vi) cooling the composition until the mixture solidifies.

In step (iv), it is preferred to cool the mixture by adding ice water while homogenizing. And in step (vi), it is preferred to store the composition at room temperature for 1 hour to 2 days.

### Method and Use

The present invention also provides for a method of reducing malodour or perspiration comprising a step of topical application of the composition of the first aspect, preferably reducing perspiration. Preferably, the present invention provides for a method wherein the composition of the first aspect is applied on the underarms. The present invention also provides for a method wherein topical application of the composition of the first aspect reduces malodour or perspiration from the surface of the human body. The method in accordance with the invention is preferably non-therapeutic. By non-therapeutic is meant that the method is cosmetic in nature.

The invention also provides for use of the composition of the first aspect for reduction of bodily malodour or perspiration, preferably perspiration. The use in accordance with the invention is preferably non-therapeutic in nature, more preferably cosmetic in nature.

The invention will now be demonstrated with the help of the following non-limiting examples.

### EXAMPLES

### Materials

TABLE 1

| | | | Melting | |
|---|---|---|---|---|
| | | | point | HLB |
| Trade name | Chemical ingredient | Supplier | (° C.) | value |
| Hostacerin ® | Polyglyceryl-2 Stearate | Clariant | NA | 4.5 |
| DGMS | | | | |
| SYNCROWAX | Tribehenin (having a | Croda | 57-62 | NA |
| HRC-PA-(RB) | straight alkyl chain with | | | |
| | 22 carbon atoms) | | | |
| CRODESTA ™ | sucrose stearate | Croda | 51-62 | 14.5 |
| F160-PW-(RB) | | | | |
| Hydrogenated | Hydrogenated soybean | Wilmar | 52 | NA |
| soybean oil | oil (having a straight | | | |
| | alkyl chain with 18 | | | |
| | carbon atoms) | | | |

### Example 1

This example demonstrates the impact on the formation of stick composition from the different combination of surfactant with the presence of the specific organic compound of the present invention. In the following examples all percentages are by weight, unless otherwise indicated.

The compositions indicated in Table-2 were prepared by following the steps:

1) Aqueous phase: water-soluble ingredients and emulsifier with HLB value >10.5 (if there is any) was dissolved in 70-75° C. DI water in a beaker by homogenizing at 11400 rpm for 5 min.

2) Oil phase: lipophilic material and nonionic emulsifier with HLB value 10.5 or lower were mixed together and then melted at 70-75° C. The melted oil phase was kept at 70-75° C. in water bath until further use.

3) The melted oil phase was added into the aqueous phase slowly while being homogenized at 11,400 rpm, then homogenized for another 5 mins.

4) Remove the heater, then add ice water (0° C., the mixture of DI water and glycerol) into above emulsion quickly when homogenizing, followed by homogenizing at 11,400 rpm for 10 seconds.

5) Other ingredients were added after the bulk being cooled to 40° C.

TABLE 2

| | Samples | | | | | | |
|---|---|---|---|---|---|---|---|
| Ingredient wt % | A | B | C | D | 1 | 2 | 3 |
| Tribehenin | 12 | 12 | 12 | 12 | 12 | 12 | 12 |
| Polyglyceryl-2 Stearate | 0 | 1 | 4 | 5 | 2 | 3.5 | 3 |
| Sucrose stearate | 5 | 4 | 1 | 0 | 3 | 1.5 | 2 |
| Xanthan gum | 0.1 | 0.1 | 0.1 | 0.1 | 0.1 | 0.1 | 0.1 |
| Glycerol | 0.5 | 0.5 | 0.5 | 0.5 | 0.5 | 0.5 | 0.5 |
| Preservative | 0.55 | 0.55 | 0.55 | 0.55 | 0.55 | 0.55 | 0.55 |
| Fragrance | 1 | 1 | 1 | 1 | 1 | 1 | 1 |
| Deionised water | To 100 | To 100 | To 100 | To 100 | To 100 | To 100 | To 100 |
| Average HLB value of | 14.5 | 12.5 | 6.5 | 4.5 | 10.5 | 7.5 | 8.5 |
| emulsifier mixture | | | | | | | |

### Method of Measurement

The samples in Table-2 were kept at room temperature for 7 days after preparation and then the hardness of the samples was measured by a texture analyzer (Stable Micro Systems). A needle penetrated the solid emulsion for 10 mm with a speed of 2 mm/s, and the force curve was recorded as the hardness curve. The maximum force (kg) of the curve is determined as the hardness.

Results are summarised in Table-3.

TABLE 3

| Samples | A | B | C | D | 1 | 2 | 3 |
|---|---|---|---|---|---|---|---|
| Hardness (kg) | 0.005 | 0.012 | 0.004 | 0.004 | 0.047 | 0.051 | 0.114 |

The results (shown in the Table-3) demonstrate that compositions as per the invention (Sample 1 to 3) is capable of forming stick composition with acceptable hardness (at least 0.015 kg), while compositions outside the invention (Sample A to D) do not form stick composition.

### Example 2

This example demonstrates the impact on the formation of stick composition from the different dosage of the specific organic compound of the present invention in the composition. In the following examples all percentages are by weight, unless otherwise indicated.

The compositions indicated in Table-4 were prepared by the process as disclosed above in Example 1.

TABLE 4

| | Samples | | |
|---|---|---|---|
| Ingredient wt % | E | 4 | 5 |
| Tribehenin | 6 | 10 | 12 |
| Polyglyceryl-2 Stearate | 3.5 | 3.5 | 3.5 |
| Sucrose stearate | 1.5 | 1.5 | 1.5 |
| Xanthan gum | 0.1 | 0.1 | 0.1 |
| Glycerol | 0.5 | 0.5 | 0.5 |
| Preservative | 0.55 | 0.55 | 0.55 |
| Fragrance | 1 | 1 | 1 |
| Deionised water | To 100 | To 100 | To 100 |
| Average HLB value of | 7.5 | 7.5 | 7.5 |
| emulsifier mixture | | | |

The hardness of samples in Table-4 after being kept at room temperature for 7 days was measured by the same method as disclosed in Example 1.

Results are summarised in Table-5

TABLE 5

| Samples | E | 4 | 5 |
|---|---|---|---|
| Hardness (kg) | 0.004 | 0.020 | 0.087 |

The results (shown in the Table-5) demonstrate that compositions as per the invention (Sample 4 and 5) is capable of forming stick composition with acceptable hardness (at least 0.015 kg), while compositions outside the invention (Sample E) does not form stick composition.

### Example 3

This example demonstrates the composition of the present invention is capable of forming aggregation/agglomeration when they contact with aqueous saline media. All ingredients are expressed by weight percent of the total formulation, and as level of active ingredient.

The composition indicated in Table-6 was prepared by the process as disclosed above in Example 1.

TABLE 6

| | Sample |
|---|---|
| Ingredient wt % | 6 |
| Tribehenin | 12 |
| Polyglyceryl-2 Stearate | 3.5 |
| Sucrose stearate | 1.5 |
| Xanthan gum | 0.1 |
| Glycerol | 0.5 |
| Preservative | 0.55 |
| Fragrance | 1 |
| Deionised water | To 100 |
| Average HLB value of emulsifier | 7.5 |
| mixture | |

The composition of model ionic sweat (pH 6.9) is as given below in Table-7:

TABLE 7

| Ingredient | wt % of total |
|---|---|
| Potassium Chloride | 0.0373 |
| Sodium Bicarbonate | 0.2025 |
| Sodium Chloride | 0.2098 |
| Ammonium Chloride | 0.0107 |
| Calcium Chloride | 0.0222 |
| Lactic Acid | 0.0901 |
| Urea | 0.0018 |
| Water | 99.4256 |

### Testing on a Narrow Capillary Apparatus

An experiment was arranged using the device as disclosed in the International Application WO2018099931 A1 (Unilever).

The device has a 20×20 μm channel. Before use, the channel was filled with model ionic sweat. The surface tension of the model ionic sweat is strong and prevents its flowing in the capillary. About 0.02 g of concerned composition in Table-2, was placed at one end of the channel. When the composition contacts with the model ionic sweat, the composition of the present invention is believed to turn into something like an aggregation/agglomeration. This phenomenon simulates the manner in which an antiperspirant active agent forms aggregation/agglomeration thereby provides antiperspirant benefit in the underarm region.

The whole process was monitored under optical microscope (Leica™ DM 2500P).

The aggregation/agglomeration formation of the compositions at the end of the channel was found after around 5 minutes.

It indicates that compositions as per the invention can form an aggregation/agglomeration under the test conditions disclosed earlier, which further indicates its ability to form aggregation/agglomeration when used in an antiperspirant composition,

### Example 4

This example demonstrates the impact on the formation of stick composition from the lipophilic material having a straight alkyl chain comprising different number of carbon atoms in the composition. In the following examples all percentages are by weight, unless otherwise indicated.

The compositions indicated in Table-8 were prepared by the process as disclosed above in Example 1.

TABLE 8

Samples

| Ingredient wt % | F | 4 |
|---|---|---|
| Hydrogenated soybean | 10 | — |
| oil (having a straight | | |
| alkyl chain with 18 | | |
| carbon atoms) | | |
| Tribehenin (having a | — | 10 |
| straight alkyl chain with | | |
| 22 carbon atoms) | | |
| Polyglyceryl-2 Stearate | 3.5 | 3.5 |
| Sucrose stearate | 1.5 | 1.5 |
| Xanthan gum | 0.1 | 0.1 |
| Glycerol | 0.5 | 0.5 |
| Preservative | 0.55 | 0.55 |
| Fragrance | 1 | 1 |
| Deionised water | To 100 | To 100 |
| Average HLB value of | 7.5 | 7.5 |
| emulsifier mixture | | |

The hardness of samples in Table-8 after being kept at room temperature for 7 days was measured by the same method as disclosed in Example 1.

Results are summarised in Table-9

TABLE 9

| Samples | F | 4 |
|---|---|---|
| Hardness (kg) | 0.0027 | 0.020 |

The results (shown in the Table-9) demonstrate that compositions as per the invention (Sample 4) can form stick composition with acceptable hardness (at least 0.015 kg), while compositions outside the invention (Sample F) does not form desired stick composition.

1. A deodorant or an antiperspirant stick composition in the form of an oil-in-water dispersion or emulsion, comprising:
i) at least one lipophilic material having a melting point higher than 50° C.;
ii) at least one nonionic emulsifier having a HLB value from 1 to 11 within an oil-in-water emulsifier mixture with an average HLB value in the range of from 6.8 to 11;
iii) an aqueous cosmetically acceptable carrier;
wherein the composition comprises from 8% to 20% of the lipophilic material by weight of the composition; wherein the composition comprises higher than 50% of water by weight of the composition; wherein the lipophilic material has a straight alkyl chain comprising at least 20 carbon atoms; wherein the nonionic emulsifier is a polyglyceryl ester selected from PEG-25 hydrogenated castor oil, glyceryl laurate, glyceryl stearate, polyglyceryl-2-distearate, polyglyceryl-2-stearate and polyglyceryl-3 distearate;
and further wherein the composition comprises an additional nonionic emulsifier that is sucrose stearate.

2. The stick composition as claimed in claim 1, wherein the nonionic emulsifier is polyglyceryl-2 stearate.

3. The stick composition as claimed in claim 1, wherein the composition comprises at least 2 wt % of the nonionic emulsifier by weight of the composition.

4. (canceled)

5. (canceled)

6. The stick composition as claimed in claim 1, wherein the composition comprises from 0.5 to 20 wt % of the sucrose stearate by weight of the composition.

7. The stick composition as claimed in claim 1, wherein the lipophilic material has a melting point of at least 55° C.

8. The stick composition as claimed in claim 1, wherein the lipophilic material is a fatty alcohol, a fatty acid, a fatty ester, a wax or a mixture thereof.

9. (canceled)

10. The stick composition as claimed in claim 1, wherein the composition comprises from 60 to 90 wt % of water by weight of the composition.

11. A method of manufacture of the deodorant or antiperspirant stick composition as claimed in claim 1 comprising the steps of:
(i) making an aqueous phase by blending together water and water-soluble ingredients and heating the aqueous phase to a temperature of 60° C. or greater;
(ii) forming an oil phase comprising a lipophilic material and nonionic emulsifier and heating the aqueous phase to a temperature of 70° C. or greater;
(iii) with the aqueous phase and oil phase at the temperatures as indicated in steps (i) and (ii) respectively, mixing these phases together;
(iv) cooling the mixture to 50° C. or below while homogenizing;
(v) filling the resultant mixture into a dispenser whilst it is still mobile; and
(vi) cooling the composition until the mixture solidifies.

12. A method of reducing malodour or perspiration comprising a step of topical application of the deodorant or antiperspirant stick as claimed in claim 1.

13. The method as claimed in claim 12, wherein the composition is applied on the underarms.

14. Use of the deodorant or antiperspirant stick as claimed in claim 1 for reduction of bodily malodour or perspiration.

15. The stick composition as claimed in claim 1, wherein the composition comprises from 2 to 20 wt % of the nonionic emulsifier by weight of the composition.

16. The stick composition as claimed in claim 1, wherein the composition comprises from 60 to 85 wt % of water by weight of the composition.

17. The stick composition as claimed in claim 1, wherein the composition comprises from 12 to 20 wt % of the lipophilic material by weight of the composition.

18. The stick composition as claimed in claim 1, wherein the oil-in-water emulsifier mixture has an average HLB value in the range of from 7 to 11.

19. The stick composition as claimed in claim 1, wherein the composition has a hardness of at least 0.015 kg.

20. The stick composition as claimed in claim 1, wherein the composition has a hardness of at least 0.02 kg.
